(19) United States
(12) Patent Application Publication
(10) Pub. No.: US 2022/0257949 A1
(43) Pub. Date: Aug. 18, 2022

(54) FEEDBACK SYSTEM TO AUTOMATE DRUG TITRATION AND STIMULATION THERAPY

(71) Applicant: Medtronic, Inc., Minneapolis, MN (US)

(72) Inventors: Michael E. Winek, Minneapolis, MN (US); Lauren E. Barth, Minneapolis, MN (US)

(21) Appl. No.: 17/177,607

(22) Filed: Feb. 17, 2021

(51) Int. Cl. A61N 1/36; A61M 5/142; A61M 5/172; A61N 1/372; G16H 40/67; G16H 20/17; G16H 20/30; G16H 10/20; G16H 10/60; G16H 50/70; G16H 20/70; G16H 50/20; G06N 20/00

(52) U.S. Cl. CPC A61N 1/36132; A61M 2205/3303; A61M 5/1723; A61N 1/37247; A61N 1/36139; A61N 1/36082; A61N 1/36067; G16H 40/67; G16H 20/17; G16H 20/30; G16H 10/20; G16H 10/60; G16H 50/70; G16H 20/70; G16H 50/20; G06N 20/00; A61M 2205/502; A61M 5/14276

(57) ABSTRACT

A medical system, including an implantable medical device configured to administer therapy to a patient according to a programmed therapy delivery profile for treatment of a disease, disorder or condition, and a user interface configured to collect qualitative patient feedback regarding one or more symptoms related to the disease, disorder or condition, wherein the system is configured to use the collected qualitative patient feedback to personalize the programmed therapy delivery profile for improved treatment of the disease, disorder or condition.

### TECHNICAL FIELD

The present technology is generally related to implantable medical devices, and more particularly to implantable medical pumps, ports and neurostimulation devices for managing the delivery and dispensation of prescribed therapeutic agents.

### BACKGROUND

Implantable medical devices, such as implantable pumps, ports and neurostimulation devices are useful in managing the delivery and dispensation of a prescribed therapy, whether that therapy is in the form of infusates (e.g., medicaments and other fluid or fluid like substances) or electrical nerve stimulation (e.g., deep brain stimulation (DBS), spinal cord stimulation (SCS), or the stimulation of other portions of the central or peripheral nervous system). Such implantable medical devices provide the advantage of delivery and dispensation of prescribed therapy in precise volume- and time-controlled doses, often on an established or prescribed delivery schedule. As such, implantable medical devices are particularly useful for treating diseases and disorders that require regular or chronic (e.g., long-term) pharmacological intervention. Further, such implantable medical devices avoid the problem of patient noncompliance, namely the patient failing to take the prescribed therapy as instructed.

Such implantable medical devices are typically implanted at a location within the body of the patient (e.g., in a subcutaneous region of the lower abdomen, etc.). Implantable pumps and ports often include a catheter configured as a flexible tube to deliver infusate to a selected delivery site within the patient (e.g., specific areas within the vasculatures or nervous system, including the subarachnoid, epidural, intrathecal, and intracranial spaces). Neurostimulation devices often include a lead including one or more stimulation electrodes configured to deliver electrical pulses to targeted nerves within the central or peripheral nervous system of the patient.

Such implantable pumps, ports and neurostimulation devices have proven effective for a wide variety of treatments, including tremor, spasticity, multiple sclerosis, Alzheimer's disease, Parkinson's disease, amyotrophic lateral sclerosis (ALS), Huntington's disease, cancer, epilepsy, chronic pain, urinary or fecal incontinence, sexual dysfunction, obesity, and gastroparesis, to name just a few. As the use of such implantable pumps, ports and neurostimulation devices expands to the treatment of other types of diseases, disorder and conditions, further improvements in obtaining feedback regarding the effectiveness of a prescribed therapy in the treatment of the particular disease, disorder or condition is desired.

### SUMMARY OF THE DISCLOSURE

The techniques of this disclosure generally relate to implantable medical devices, systems and methods configured to use qualitative patient feedback to make predictions or suggestions for therapy parameters in the treatment of a disease, disorder or condition. In some embodiments, the implantable medical device, system or method can be configured to treat a disease, disorder or condition having one or more subjective symptoms. The device, system or method can actively engage with the patient or passively observe the patient to gather data regarding the one or more subjective symptoms for use in tailoring or personalizing a prescribed therapy profile to the patient. For example, in some embodiments, the implantable medical device, system or method can be used in the treatment of one or more psychiatric applications (e.g., treatment of depression, anxiety disorders, schizophrenia, eating disorders, addictive behaviors, etc.), chronic pain, bodily movement disorders (e.g., Parkinson's disease, progressive supranuclear palsy, multiple system atrophy, etc.) or other diseases, disorders or conditions with symptoms, including feelings, sensations and perceptions, which do not necessarily correlate with observable objective physiological conditions of the patient. In some embodiments, the devices, systems and methods can be utilized to increase the efficacy and management of current therapies, as well as to ease the adoption of possible new therapies. In some embodiment, the devices, systems and methods can alert users to a potential failure or system malfunction (e.g., a drastic change in symptoms may be indicative of a break in a lead or catheter fracture, which may warrant further evaluation by a health care provider).

One embodiment of the present disclosure provides a medical system including an implantable medical device configured to administer therapy to a patient according to a programmed therapy delivery profile for treatment of a disease, disorder or condition, and a user interface configured to collect qualitative patient feedback regarding one or more symptoms related to the disease, disorder or condition, wherein the system is configured to use the collected qualitative patient feedback to personalize the programmed therapy delivery profile for improved treatment of the disease, disorder or condition.

In one embodiment, the implantable medical device comprises at least one of an implantable pump or neurostimulation device. In one embodiment, the user interface is presented via an external programmer in wireless communication with the implantable medical device. In one embodiment, the external programmer comprises at least one of a cellular telephone, tablet, computer, or dedicated implantable device programmer. In one embodiment, the qualitative patient feedback comprises observations regarding at least one of feelings, sensations or perceptions specific to the patient. In one embodiment, the qualitative patient feedback regarding the one or more symptoms is at least partially gathered via one or more questionnaires presented to the patient on the user interface. In one embodiment, the system is configured to detect at least one of changes or trends in the qualitative patient feedback over a period of time. In one embodiment, the system further includes one or more sensors configured to observe objective physiological criteria. In one embodiment, the system further includes one or more servers configured to communicate with at least one of the implantable medical device and user interface.

Another embodiment of the present disclosure provides a medical system for the treatment of one or more psychiatric conditions, a medical system configured to collect qualitative patient feedback relating to symptoms of the one or more psychiatric conditions for modification of a delivered therapy protocol in treatment of one or more psychiatric conditions. The medical system can include an implantable medical device configured to administer therapy according to a programmed delivery therapy protocol for the treatment of one or more psychiatric conditions, and an external programmer in communication with the implantable medical device, the external programmer comprising a user interface configured to collect qualitative patient feedback relating to symptoms of the one or more psychiatric conditions.

In one embodiment, the implantable medical device comprises at least one of an implantable pump or neurostimulation device. In one embodiment, the qualitative patient feedback comprises observations regarding at least one of feelings, sensations or perceptions specific to the patient. In one embodiment, the system is configured to identify at least one of changes or trends in the one or more symptoms relating to the disease, disorder or condition over a period of time. In one embodiment, the one or more symptoms relating to the disease, disorder or condition comprise at least one of depression, anxiety, or a psychotic episode.

Another embodiment of the present disclosure provides a medical system, including an implantable medical device configured to administer therapy to a patient according to a programmed therapy delivery profile for treatment of a disease, disorder or condition, a user interface configured to passively collect qualitative patient feedback regarding one or more symptoms related to the disease, disorder or condition, and a neural network configured to analyze the qualitative patient feedback to infer a change in one or more symptoms relating to the disease, disorder or condition, and to modify the programmed therapy delivery profile for improved treatment of the disease, disorder or condition.

In one embodiment, the qualitative patient feedback comprises at least one of monitored facial expressions, tone of voice, written text, internet search terms, or a combination thereof. In one embodiment, the neural network is configured to learn to recognize one or more symptoms relating to the disease, disorder or condition specific to an individual patient. In one embodiment, the system is configured to identify at least one of changes or trends in the one or more symptoms relating to the disease, disorder or condition over a period of time. In one embodiment, the system is configured to automatically modify the programmed therapy delivery profile, while monitoring for a change in the one or more symptoms relating to the disease, disorder or condition. In one embodiment, the one or more symptoms relating to the disease, disorder or condition comprise at least one of physical pain, depression, anxiety, a psychotic episode, or limited mobility.

The details of one or more aspects of the disclosure are set forth in the accompanying drawings and the description below. Other features, objects, and advantages of the techniques described in this disclosure will be apparent from the description in the drawings, and from the claims.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a schematic view depicting a medical system configured to gather data regarding patient specific symptoms of a disease, disorder or condition in order to prescribe or personalize a prescription therapy profile for treatment of the disease, disorder or condition, in accordance with an embodiment of the disclosure.

FIG. 2 is a partial, cross-sectional view depicting a portion of a medical system configured to gather data regarding patient symptoms for personalization of a prescription therapy profile, in accordance with an embodiment of the disclosure.

FIG. 3 is a block diagram of an implantable device configured modify a programmed therapy delivery profile for improved treatment of a disease, disorder or condition in response to data gathered regarding symptoms of the disease, disorder or condition, in accordance with an embodiment of the disclosure.

FIG. 4 is a flowchart depicting a method of gathering data regarding patient specific symptoms of a disease, disorder or condition in order to prescribe or personalize a prescription therapy profile for treatment of the disease, disorder or condition, in accordance with an embodiment of the disclosure.

FIG. 5A is a diagram depicting a neural network configured to analyze data regarding patient symptoms for personalization of a prescription therapy profile, in accordance with an embodiment of the disclosure.

FIG. 5B depicts a single neuron within the neural network of FIG. 5A, in accordance with an embodiment of the disclosure.

FIG. 6 is a perspective view depicting a medical system configured to recognize changes in facial expression or mood for personalization of a therapy profile, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

Various example embodiments of implantable medical devices, systems and methods are described herein for personalizing therapy delivery profiles for a disease, disorder or condition based on qualitative feedback regarding one or more subjective symptoms of the disease, ailment or condition, wherein like reference numerals represent like parts and assemblies throughout the several views. Although specific examples of implantable medical pumps and neurosimulation devices are provided, it is to be appreciated that the concepts disclosed herein are extendable to other types of implantable devices. Further, while various treatments for chronic pain, tremor, spasticity and psychiatric conditions are provided, embodiments of the present disclosure can be used to treat a host of other diseases, disorders and conditions. It is also to be appreciated that the term “clinician” refers to any individual that can prescribe and/or program a therapeutic regimen with any of the example embodiments described herein or alternative combinations thereof. Similarly, the term “patient” or “subject,” as used herein, is to be understood to refer to an individual or object in which the therapy is to occur, whether human, animal, or inanimate. Various descriptions are made herein, for the sake of convenience, with respect to the procedures being performed by a clinician on a patient or subject (the involved parties collectively referred to as a “user” or “users”) while the disclosure is not limited in this respect.

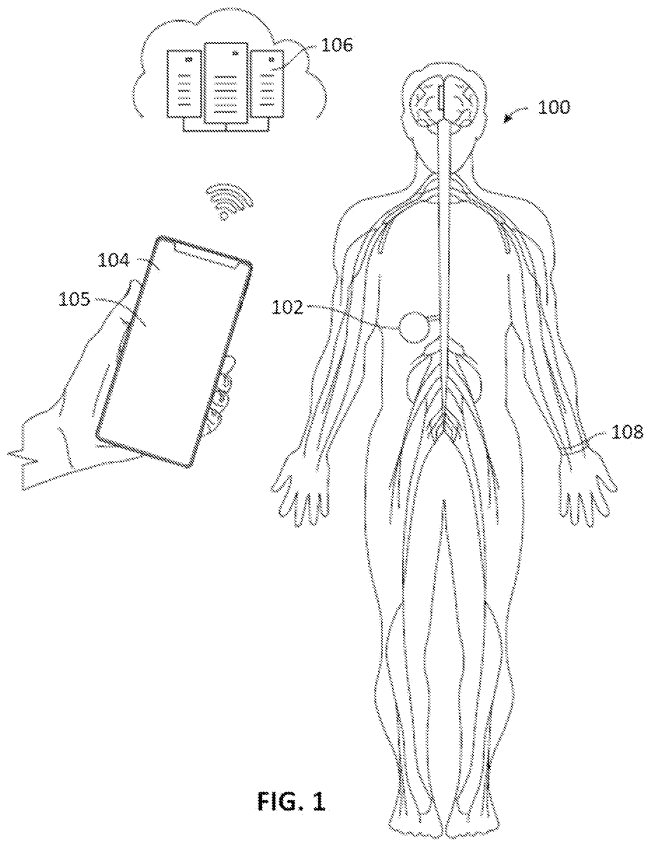

Referring to FIG. 1, a medical system 100 configured to gather data regarding patient specific symptoms of a disease, disorder or condition in order to prescribe or personalize a prescription therapy profile for treatment of the disease, disorder or condition is depicted in accordance with an embodiment of the disclosure. The medical system 100 can include an implantable medical device 102, for example in the form of an implantable pump/port or neurostimulator device. As depicted, the implantable device 102 can be implanted within the body of the patient (e.g., in an interior torso cavity, cranially, etc.) for targeted delivery of therapy to a specific site within the patient (e.g., within an intrathecal space, or intracranial space, pulmonary artery, etc.). In some embodiments, the implantable device 102 can be placed subcutaneously, and can be held in position by one or more sutures or other retaining features.

In some embodiments, the medical system 100 can further include an optional external programmer 104 and optional server 106 (e.g., a cloud-based server or network of servers) configured to communicate with the implantable device 102. In some embodiments, the external programmer 104 can include a user interface 105 configured to receive user input, as well as to potentially passively observe behavior patterns of the patient. In some embodiments, the programmer 104 can be a handheld, wireless portable computing device, such as a cellular telephone, tablet, dedicated implantable device programmer, or the like. Further, in some embodiments, the medical system 100 can include one or more external physiological sensors 108, which can be in communication with the implantable device 102, optional external programmer 104, and optional server 106. In one embodiment, one or more physiological sensors 108 can be incorporated into the implantable device 102 or external programmer 104. In one embodiment, a physiological sensor 108 can be worn by the patient (e.g., a smart watch, wristband tracker, sensors embedded in clothing, etc.), carried by the patient (e.g., a smart phone, mobile computing device, etc.), or positioned in proximity to the patient (e.g., a stationary monitor, etc.). Examples of physiological sensors 108 include a heart rate monitor, pulse oximeter, respiratory sensor, perspiration sensor, posture orientation sensor, motion sensor, accelerometer, or the like.

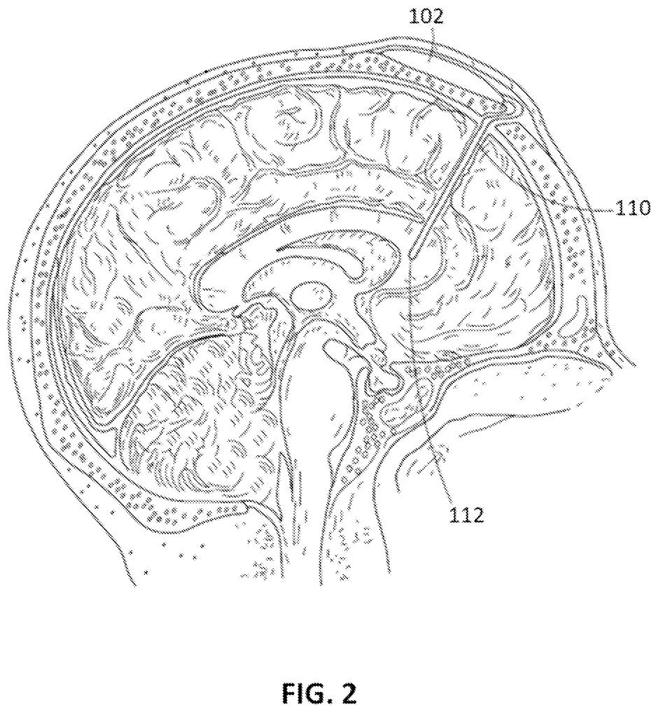

With additional reference to FIG. 2, an alternative implantable device 102 configured to gather data regarding patient specific symptoms of a disease, disorder or condition in order to prescribe or personalize a prescription therapy profile for treatment of the disease, disorder or condition, is depicted in accordance with an embodiment of the disclosure. In some embodiments, the implantable device 102 can include a flexible catheter or electrical lead 110 configured to administer therapy (e.g., infusate or electrical stimulation) via a distal tip 112 to a target site within the body of the patient.

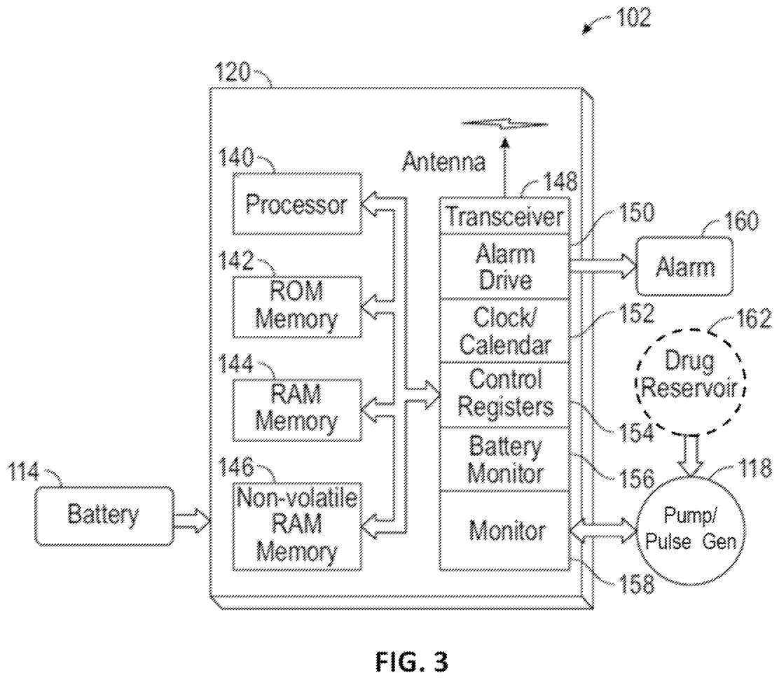

Referring to FIG. 3, a block diagram of an implantable device 102 configured to gather data regarding patient specific symptoms of a disease, disorder or condition in order to prescribe or personalize a prescription therapy profile for treatment of the disease, disorder or condition is depicted in accordance with an embodiment of the disclosure. In some embodiments, the implantable device 100 can include a computing device 120, for example carried in a housing. For example, in some embodiments, the housing can be constructed of a material that is biocompatible and hermetically sealed, such as titanium, tantalum, stainless steel, plastic, ceramic, or the like.

The computing device 120 can be in communication with a power source 114 and a pump or pulse generator 118. The power source 114 can be a battery, such as a rechargeable lithium-ion battery; although other power sources are also contemplated including inductively powered or charged sources, enabling a contactless source of power to be transmitted to the implantable device 102. In some embodiments, the power source 114, which can be monitored via a battery monitor 156, can selectively operate the pump/pulse generator 118 and computing device 120. Control of the pump/pulse generator 118 can be directed by a monitor element 158.

The computing device 120 can include a processor 140, memory 142, 144 & 146, and transceiver circuitry 148. In one embodiment, the processor 140 can be a microprocessor, logic circuit, Application-Specific Integrated Circuit (ASIC) state machine, gate array, controller, or the like. The computing device 120 can generally be configured to control the delivery or administration of a prescribed treatment protocol according to programmed parameters or a prescribed therapy. The programmed parameters or prescribed therapy can be stored in the memory 142, 144 & 146 for specific implementation by a control register 154. A clock/calendar element 152 can maintain system timing for the computing device 120. In one embodiment, an alarm drive 150 can be configured to activate one or more notification, alert or alarm features, such as an illuminated, auditory or vibratory alarm 160.

The transceiver circuitry 148 can be configured to receive information from and transmit information to the one or more physiological sensors 108, external programmer 104, and server 106. The implantable device 102 can be configured to receive programmed parameters and other updates from the external programmer 104, which can communicate with the implantable device 102 through well-known techniques such as wireless telemetry, Bluetooth, or one or more proprietary communication schemes (e.g., Tel-M, Tel-C, etc.). In some embodiments, the external programmer 104 can be configured for exclusive communication with one or more implantable device 102. In other embodiments, the external programmer 104 can be any computing platform, such as a mobile phone, tablet or personal computer. In some embodiments, the implantable device 102 and external programmer 104 can further be in communication with a cloud-based server 104, configured to receive, store and transmit information, such as program parameters, treatment protocols, drug libraries, and patient information, as well as to receive and store data recorded by the implantable device 102.

In embodiments of the implantable device 102 including a pump 118, the pump 118 can be in fluid communication with a drug reservoir 162 and can be in electrical communication with the computing device 120. The pump 118 can be any pump sufficient for infusing fluid to the patient, such as a peristaltic pump, piston pump, a pump powered by a stepper motor or rotary motor, a pump powered by an AC motor, a pump powered by a DC motor, electrostatic diaphragm, piezoelectric motor, solenoid, shape memory alloy, or the like. In embodiments of the implantable device including a pulse generator 118, the pulse generator 118 can be configured to generate and distribute prescribed stimulation waveform patterns or trains (e.g., through lead 110), to selectively stimulate the targeted tissue region. Other configurations of implantable device 102 are also contemplated.

With continued reference to FIGS. 1-3, in some embodiments, the medical system 100 can be configured to actively request feedback from the patient or passively observe the patient to collect data regarding one or more subjective symptoms, for the purpose of tailoring or personalizing a prescribed therapy profile of administered infusate or electrical stimulation in the treatment of a disease, disorder or condition. In embodiments, the disease, disorder or condition can be one in which symptoms of the disease, disorder or condition are subjective, that is the symptoms may primarily manifest through feelings, sensations and perceptions. Examples of such diseases, disorders and conditions include chronic pain, the symptoms of which are only indirectly physiologically measurable (e.g., via an increase in heart rate, perspiration, etc.), or various psychiatric conditions (e.g., depression, anxiety disorders, schizophrenia, eating disorders, addictive behaviors, etc.), which again may not be directly correlated with changes to objective physiological criteria (e.g., heart rate, respiratory rate, etc.).

In such cases, the personalized tailoring of specific treatment protocols may rely heavily on feedback or questionnaire base qualification of the disease, disorder or condition state in order to titrate drugs or adjust an electrical stimulation profile. In some embodiments, these questionnaires can be incorporated into an automated system, such as the external programmer 104 to adjust therapeutic dosing or to track changes and provide recommendations to clinicians on therapy delivery profiles, including improvements in the timing, quantity and duration of the therapeutic delivery. Beyond improvements to therapy delivery profiles, embodiments of the present disclosure also enable a more rapid assessment of an increased patient tolerance to the therapy, loss of sensation of the target site, medical device malfunctions, and the like.

In embodiments, the processor 140 of the implantable medical device 102 can be configured to track patient specific subjective feedback along with a history of the delivery therapy and any patient-initiated boluses or treatments, in an effort to identify trends, as well as any correlations between changes in patient specific subjective feedback and the delivered therapy. Various statistical techniques can be used in the identification of trends and correlations between observation of symptoms and delivery profiles. In some embodiments, the user interface 105 may be configured to prompt a patient to complete one or more questionnaires related to a disease, disorder or condition on a periodic basis. Examples of questionnaires related to the treatment of psychiatric conditions can include: bipolar spectrum diagnostic scale (BSDS); mood disorder questionnaire (MDQ); Hamilton depression rating scale; Montgomery-Asberg depression rating scale; Raskin depression rating scale; Beck depression inventory; geriatric depression scale (GDS); Zung self-rating depression scale; patient health questionnaire (PHW); positive and negative symptoms scale (PANSS); scale for assessment of positive symptoms (SAPS); scale for assessment of negative symptoms (SANS); negative symptom assessment-16 (NSA_16); clinical global impression schizophrenia (CGI-SCH); clinical assessment interview for negative symptoms (CAINS); brief negative symptoms scale (BNSS); and the like.

Examples of questionnaires related to the treatment of chronic pain can include: McGill pain questionnaire (MDQ); Wong Baker faces pain scale; FLACC scale; COMFORT scale; color analog scale; Mankoski pain scale; brief pain inventory; descriptive differential scale of pain intensity; and the like. Examples of questionnaires related to movement disorders can include: movement disorder society ranking scale; craniocervical dystonia questionnaire (CDQ-24); Parkinson's disease quality of life questionnaire (PDQL); and the like. Other measurable inputs, for example measurable by one or more physiological sensors 108, can include body weight, body temperature, awake/sleep hours, activity levels, etc.

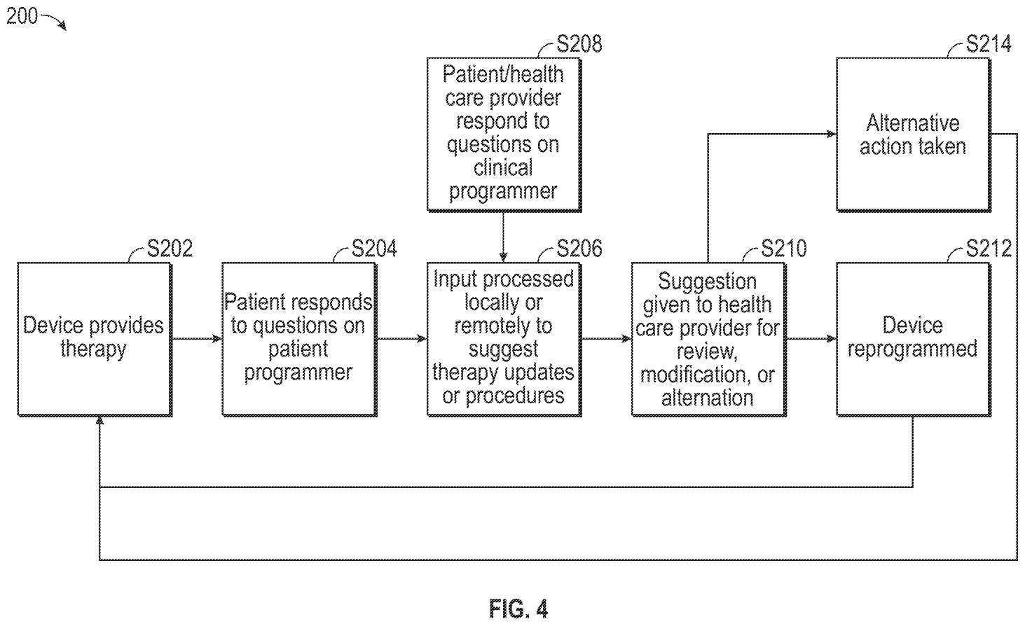

Referring to FIG. 4, a method 200 of gathering data regarding patient specific symptoms of a disease, disorder or condition in order to prescribe or personalize a prescription therapy profile for treatment of the disease, disorder or condition is depicted in accordance with an embodiment of the disclosure. At S202, the medical system 100 can provide therapeutic treatment for a disease, disorder or condition. At S204, the medical system can periodically or from time to time actively engage with the patient (e.g., via a questionnaire, query and response, or the like), or passively observe the patient to obtain data regarding one or more subjective symptoms of the disease, disorder or condition.

At S206, the medical system 100 can process collected data related to the one or more subjective symptoms (e.g., either locally within the implantable device 102 or external programmer 104, or remotely via the server 106) to suggest therapy updates or procedures for improved treatment of the disease, disorder or condition. For example, in some embodiments, the medical system 100 can be configured to identify trends in the observed symptoms of a patient, thereby providing an alert to clinician that changes to a therapy regimen may be warranted.

At 208, in some embodiments, the medical system 100 may request input from a clinician. In some embodiments, the clinician based input can be related to patient observations and/or expert medical knowledge pertaining to the particular disease, disorder or condition being treated. For example, in some embodiments, the clinician input can include an upper and lower limit or parameters for a therapeutic delivery profile. Such clinician input can be evaluated along with the patient collected data at S206.

At S210, the medical system 100 can suggest a review, modification or alteration of the therapy delivery profile. In some embodiments, the medical system 100 may require clinician approval prior to making changes to the treatment protocol. In other embodiments, the medical system 100 may automatically modify the treatment protocol, for example within clinician established upper and lower limits. At S212, the implantable device 102 can be programmed with the modified treatment protocol, and the process can be repeated for further refinement of the treatment protocol in an effort to minimize symptoms associated with the treated disease, disorder or condition. Where one or more upper or lower limits of the treatment protocol have been reached or a potential failure/system malfunction is detected (e.g., a break in a lead, catheter fracture, etc.), at S214, the medical system 100 can alert a clinician that one or more alternative actions may be required.

It should be understood that the individual steps used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

Accordingly, embodiments of the present disclosure enable users to actively track symptoms related to treating diseases, disorders and conditions, thereby enabling clinicians to maintain treatment levels at the appropriate risk/benefit ratio, and to make periodic changes to the treatment levels for improved treatment of the disease, disorder or condition. Embodiments of the present disclosure may be particularly useful where incorrect or less than optimal treatment doses may result in adverse decision-making by the patient. For example an increase in episodes related to a treated psychiatric condition (e.g., bipolar disorder, schizophrenia, etc.) may alert a clinician to a need to modify the patient's current treatment protocol. In some embodiments, the medical system 100 can automatically modify the treatment protocol within upper and lower limits established by a clinician, thereby enabling the medical system 100 to tune the treatment protocol to the specific needs of the patient.

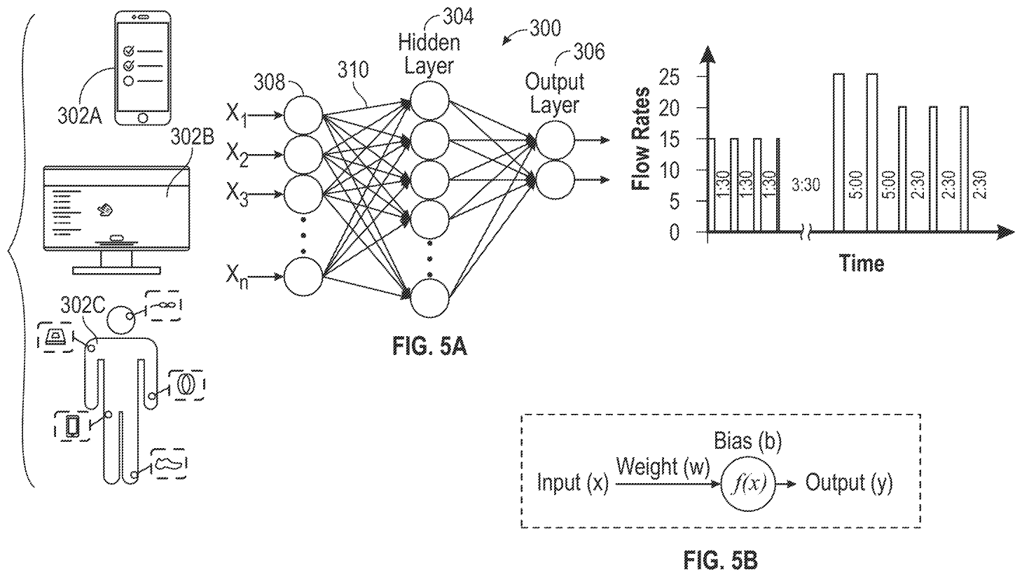

In one embodiment, the medical system 100 can utilize one or more advanced algorithms, for example via a deep learning algorithm (e.g., an artificial neural network, or the like), in an effort to personalize a prescribed therapy profile to a patient. For example, in some embodiments, the medical system 100 can be configured to evaluate a potentially large quantity of gathered data relating to symptoms of a disease, disorder or condition experienced by a patient, with the goal of improving the effect of the therapeutic delivery. For example, with reference to FIG. 5A, in one embodiment, a neural network 300, can include an input layer 302, one or more hidden layers 304, and an output layer 306. Each of the layers 302, 304, and 306 can include a corresponding plurality of neurons 308. Although only a single hidden layer 304 is depicted, it is contemplated that the neural network 300 can include multiple hidden layers 304.

The inputs for the input layer 302 can be a number between 0 and 1. Inputs to the neural network 300 can include patient questionnaire input 302A (e.g., presented on an external programmer 104), clinician related input 302B (e.g., limitations, restrictions, expert guidance), and/or feedback via one or more wearable sensors 302C (e.g., moisture probe, pressure sensor, heart rate monitor, pulse oximeter, accelerometer, etc.), wherein each of the input values for each of the inputs is scaled to a value of between 0 and 1. Further, in some embodiments, the inputs can include observations of the patient (e.g., via the external programmer 104), including monitored facial expressions, tone of voice, topics and language used in texts, e-mails, or other writings by the patient, internet search topics, etc.

Each of the neurons 308 in one layer (e.g., input layer 302) can be connected to each of the neurons 308 of the subsequent layer (e.g., hidden layer 304) via a connection 310, as such, the layers of the network can be said to be fully connected. Although it is also contemplated that the algorithm can be organized as a convolutional neural network, wherein a distinct group of input layer 302 neurons can couple to a single neuron in a hidden layer 304 via a shared weighted value.

With additional reference to FIG. 5B, each of the neurons 308 can be configured to receive one or more input values (x) and compute an output value (y). In fully connected networks, each of the neurons 308 can be assigned a bias value (b), and each of the connections 310 can be assigned a weight value (w). Collectively the weights and biases can be tuned as the neural network 300 learns how to correctly classify the input values. Each of the neurons 308 can be configured as a mathematical function, such that an output of each neuron 308 is a function of the connection weights of the collective input, and the bias of the neuron 308, according to the following relationship:

y≡w·x+b

In some embodiments, output (y) of the neuron 308 can be configured to take on any value between 0 and 1. Further, in some embodiments the output of the neuron 308 can be computed according to one of a linear function, sigmoid function, tanh function, rectified linear unit, or other function configured to generally inhibit saturation (e.g., avoid extreme output values which tend to create instability in the network 300).

An output 306 of the neural network can be a programmed therapeutic regimen or suggested modification for a therapeutic regimen. In some embodiments, the output layer 306 can include neurons 308 corresponding to a desired number of outputs of the neural network 300. For example, in one embodiment, the neural network 300 can include a plurality of output neurons dividing a period of time (e.g., 24 hrs.) into distinct increments, in which the likelihood of success of a therapeutic regimen can be indicated with an output value of between 0 and 1, such that the therapeutic regimen can be scheduled with start times, durations, concentrations or intensities, etc. based on probabilities of decreases in patient experienced symptoms associated with the treated disease, disorder or condition.

The goal of the deep learning algorithm is to tune the weights and balances of the neural network 300 until the inputs to the input layer 302 are properly mapped to the desired outputs of the output layer 306, thereby enabling the algorithm to accurately produce outputs (y) for previously unknown inputs (x). In some embodiments, the neural network 300 can rely on training data (e.g., inputs with known outputs) to properly tune the weights and balances. In tuning the neural network 300, a cost function (e.g., a quadratic cost function, cross entropy cross function, etc.) can be used to establish how close the actual output data of the output layer 306 corresponds to the known outputs of the training data. Each time the neural network 300 runs through a full training data set can be referred to as one epoch. Progressively, over the course of several epochs, the weights and balances of the neural network 300 can be tuned to iteratively minimize the cost function.

Effective tuning of the neural network 300 can be established by computing a gradient descent of the cost function, with the goal of locating a global minimum in the cost function. In some embodiments, a backpropagation algorithm can be used to compute the gradient descent of the cost function. In particular, the backpropagation algorithm computes the partial derivative of the cost function with respect to any weight (w) or bias (b) in the network 300. As a result, the backpropagation algorithm serves as a way of keeping track of small perturbations to the weights and biases as they propagate through the network, reach the output, and affect the cost. In some embodiments, changes to the weights and balances can be limited to a learning rate to prevent overfitting of the neural network 300 (e.g., making changes to the respective weights and biases so large that the cost function overshoots the global minimum). For example, in some embodiments, the learning rate can be set between about 0.03 and about 10. Additionally, in some embodiments, various methods of regularization, such as L1 and L2 regularization, can be employed as an aid in minimizing the cost function.

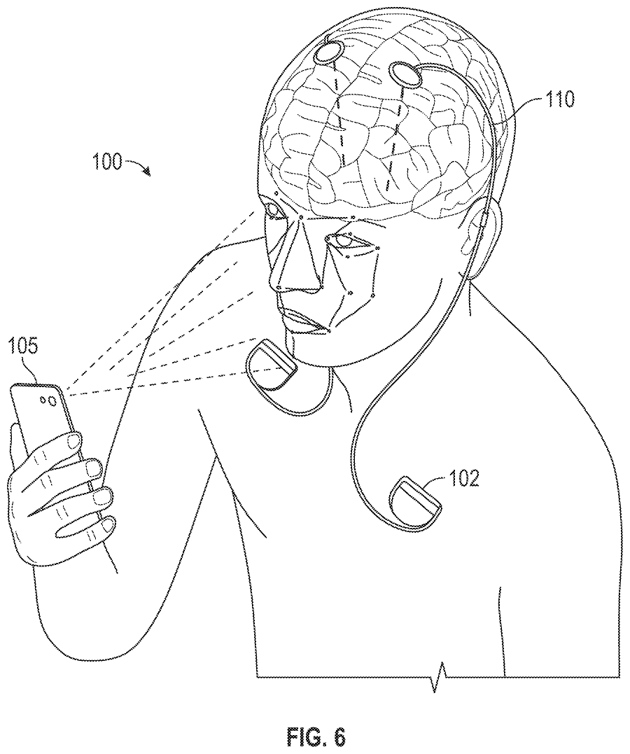

With additional reference to FIG. 6, an example use of the medical system 100 in which the external programmer 104 relies (at least partially) on facial recognition of the patient in order to assess symptoms experienced by the patient for the personalizing of a prescribed therapy profile on the implantable device 102, is depicted in accordance with an embodiment of the disclosure. Facial recognition can be particularly useful for chronic pain management (e.g., to automatically associate a patient's facial expression with a Wong Baker faces pain scale) or psychiatric conditions (e.g., monitoring a patient's face for signs of depression, anxiety, schizophrenia or the like). Similarly, the system 100 can monitor other patient activity, tone of voice, e-mail and text communications, internet searches and the like, collectively of which can be analyzed by the medical system 100 to actively monitor symptoms of a disease, disorder or condition. Moreover, in some embodiments, the medical system 100 can conduct experiments by making modifications to the prescribed therapy in an effort to identify connections between therapy protocol parameters and symptoms experienced by the patient, with the goal of minimizing adverse symptoms or at least maintaining observations of symptoms below a defined threshold.

Accordingly, in some embodiments, the medical system 100 can be configured to utilize a variety of data as an input for the cloud computing platform 106 configured to operate a deep learning algorithm for the purpose of automatically scheduling neurostimulation therapy with a goal of improving neurostimulation therapy specifically to maximize the therapeutic effect of the neurostimulation therapy.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A medical system, comprising:
an implantable medical device configured to administer therapy to a patient according to a programmed therapy delivery profile for treatment of a disease, disorder or condition; and
a user interface configured to collect qualitative patient feedback regarding one or more symptoms related to the disease, disorder or condition, wherein the system is configured to use the collected qualitative patient feedback to personalize the programmed therapy delivery profile for improved treatment of the disease, disorder or condition.

2. The medical system of claim 1, wherein the implantable medical device comprises at least one of an implantable pump or neurostimulation device.

3. The medical system of claim 1, wherein the user interface is presented via an external programmer in wireless communication with the implantable medical device.

4. The medical system of claim 3, wherein the external programmer comprises at least one of a cellular telephone, tablet, computer, or dedicated implantable device programmer.

5. The medical system of claim 1, wherein the qualitative patient feedback comprises observations regarding at least one of feelings, sensations or perceptions specific to the patient.

6. The medical system of claim 1, wherein the qualitative patient feedback regarding the one or more symptoms is at least partially gathered via one or more questionnaires presented to the patient on the user interface.

7. The medical system of claim 1, wherein the system is configured to detect at least one of changes or trends in the qualitative patient feedback over a period of time.

8. The medical system of claim 1, further comprising one or more sensors configured to observe objective physiological criteria.

9. The medical system of claim 1, further comprising one or more servers configured to communicate with at least one of the implantable medical device and user interface.

10. A medical system for the treatment of one or more psychiatric conditions, a medical system configured to collect qualitative patient feedback relating to symptoms of the one or more psychiatric conditions for modification of a delivered therapy protocol in treatment of one or more psychiatric conditions, the medical system comprising:
an implantable medical device configured to administer therapy according to a programmed delivery therapy protocol for the treatment of one or more psychiatric conditions; and
an external programmer in communication with the implantable medical device, the external programmer comprising a user interface configured to collect qualitative patient feedback relating to symptoms of the one or more psychiatric conditions.

11. The medical system of claim 10, wherein the implantable medical device comprises at least one of an implantable pump or neurostimulation device.

12. The medical system of claim 10, wherein the qualitative patient feedback comprises observations regarding at least one of feelings, sensations or perceptions specific to the patient.

13. The medical system of claim 10, wherein the system is configured to identify at least one of changes or trends in the one or more symptoms relating to the disease, disorder or condition over a period of time.

14. The medical system of claim 10, wherein the one or more symptoms relating to the disease, disorder or condition comprise at least one of depression, anxiety, or a psychotic episode.

15. A medical system, comprising:
an implantable medical device configured to administer therapy to a patient according to a programmed therapy delivery profile for treatment of a disease, disorder or condition;
a user interface configured to passively collect qualitative patient feedback regarding one or more symptoms related to the disease, disorder or condition; and
a learning algorithm configured to analyze the qualitative patient feedback to infer a change in one or more symptoms relating to the disease, disorder or condition, and to modify the programmed therapy delivery profile for improved treatment of the disease, disorder or condition.

16. The medical system of claim 15, wherein the qualitative patient feedback comprises at least one of monitored facial expressions, tone of voice, written text, internet search terms, or a combination thereof.

17. The medical system of claim 15, wherein the learning algorithm is configured to learn to recognize one or more symptoms relating to the disease, disorder or condition specific to an individual patient.

18. The medical system of claim 15, wherein the system is configured to identify at least one of changes or trends in the one or more symptoms relating to the disease, disorder or condition over a period of time.

19. The medical system of claim 15, wherein the system is configured to automatically modify the programmed therapy delivery profile, while monitoring for a change in the one or more symptoms relating to the disease, disorder or condition.

20. The medical system of claim 15, wherein the one or more symptoms relating to the disease, disorder or condition comprise at least one of physical pain, depression, anxiety, a psychotic episode, or limited mobility.
